(19) United States
(12) Patent Application Publication
(10) Pub. No.: US 2007/0156113 A1
(43) Pub. Date: Jul. 5, 2007

(54) Organ shealth for percutaneous delivery of biological and pharmacological agents

(75) Inventor: Mahender Macha, Huntingdon Valley, PA (US)

Correspondence Address: VOLPE AND KOENIG, P.C., UNITED PLAZA, SUITE 1600, 30 SOUTH 17TH STREET, PHILADELPHIA, PA, 19103, US

(73) Assignee: Heartcor, Huntingdon Valley, PA (US)

(21) Appl. No.: 11/648,975

(22) Filed: Jan. 3, 2007

Related U.S. Application Data
(60) Provisional application No. 60/757,558, filed on Jan. 3, 2006.

(51) Int. Cl. A61M 31/00

(52) U.S. Cl. 604/500

(57) ABSTRACT

A method and device provides long-term percutaneous access to the surface of the heart or other organs through the use of a conduit that delivers treatment to a sheath adjacent or surrounding the organ.

### CROSS REFERENCE TO RELATED APPLICATION

This application claims the benefit of U.S. provisional application No. 60/757,558 filed Jan. 3, 2006, which is incorporated by reference as if fully set forth.

### FIELD OF INVENTION

The invention relates generally to the field of organ disease therapy, and specifically to a medical device, system and method for the delivery of therapeutic agents to the organ.

### BACKGROUND

Organ disease and in particular, cardiovascular disease, is the leading cause of death worldwide. Medical research has led to the discovery of new approaches to the treatment of heart disease such as gene therapy, cellular therapy, pharmacologic therapy, biological therapy, and innovative medical procedures. One such intervention, gene therapy, is designed to replenish deficient proteins or molecules within the heart. Cellular therapy is designed to replace dysfunctional or dead tissue to improve function.

Many of these new therapies are found to be most effective when delivered directly to the organ of interest, and are associated with fewer potential side effect. Gene therapy, for example, delivered by adenovirus or plasmid has shown success as a potential therapeutic modality for treating cardiovascular diseases and disorders such as coronary artery disease and congestive heart failure. Of particular interest is end stage heart disease. Generally, end stage heart disease is heart disease of any origin that progressed to an end stage or an advanced form of that disease. Although the patient can be maintained on treatment, the patient is still sick, probably disabled, and generally unable to function at even limited levels of activity. For example, someone who has end stage ischemic heart disease has been suffering with that problem for many years. They will frequently have a history of multiple heart attacks and likely prior surgical intervention.

Recently, there has been increased enthusiasm for the delivery of stem cells to regenerate or replace damaged myocardium. Several techniques have been used to deliver these modalities to the heart, including trans-venous, trans-arterial (intra myocardial), trans-coronary, and epicardial. However, applications such as these are hindered by the relative inaccessibility to the heart. Further, many of these therapies, which would include pharmacologic therapies, have transient effects and could be more effective if they were delivered continuously or repeatedly, as with oral or intravenous medications. In addition, some therapies are highly toxic to other organs and directed delivery to the target organ would reduce systemic toxicity and improve efficacy. One such example, would be the delivery of imniunosuppression to solid organ transplants such as the heart, liver or kidney.

Various attempts have been made to solve these deficiencies. While traditional methods include medication routinely administered by intravenous and oral routes, recent techniques include direct access to the heart to deliver pharmacologic or biologic agents. For example, direct access to the heart can be obtained percutaneously through the femoral or jugular venous system. However, such intervention suffers drawbacks including the pain and suffering associated with the insertion of large percutaneous sheaths, the scar tissue that develops from repeated access, the morbidity of unexpected vascular injury, and the fact that direct access methods cannot be left in place chronically or over an extended period of time. There are no medical devices for the chronic administration of therapeutic agents to the heart or surface of the heart. Alternatively, surgical access to the surface of the heart may be obtained by median sternotomy, thoracotomy, thoracoscopy, or subxiphoid exposure, which may have to be performed on a repeated basis depending on several factors. These highly invasive procedures are not acceptable options for long-term access to the heart.

There are no prior devices designed to deliver therapeutic agents to the surface of the heart on a long-term basis. Thus, there is a need for an improved system, method and device for delivery of therapeutic agents to the heart on a long-term basis. In similar fashion, there is a need to provide long term direct access to other solid organs such as the liver, kidney, or bladder for chronic administration of pharmacologic or biologic therapy.

### SUMMARY

Accordingly, the method and device described herein solve these problems by providing long-term percutaneous access to the surface of the heart or other organs through the use of a conduit that delivers treatment to a sheath adjacent to or surrounding the organ.

### BRIEF DESCRIPTION OF THE DRAWINGS

FIG. 1 shows an example of the method and device used in delivering treatment to the heart.

FIG. 2 shows the device in use with a heart and subcutaneous port.

FIG. 3 shows an example of the method and device used in delivering treatment to the liver.

FIG. 4 shows an example of the method and device used in delivering treatment to the kidney.

FIG. 5 shows an example of the thoracoscopic method of delivering the device.

### DETAILED DESCRIPTION OF THE PREFERRED EMBODIMENTS

The device and method are related to the treatment of organ disease. Although most of the examples are discussed in the context of the treatment of heart disease, this preferred treatment description is by no means limiting.

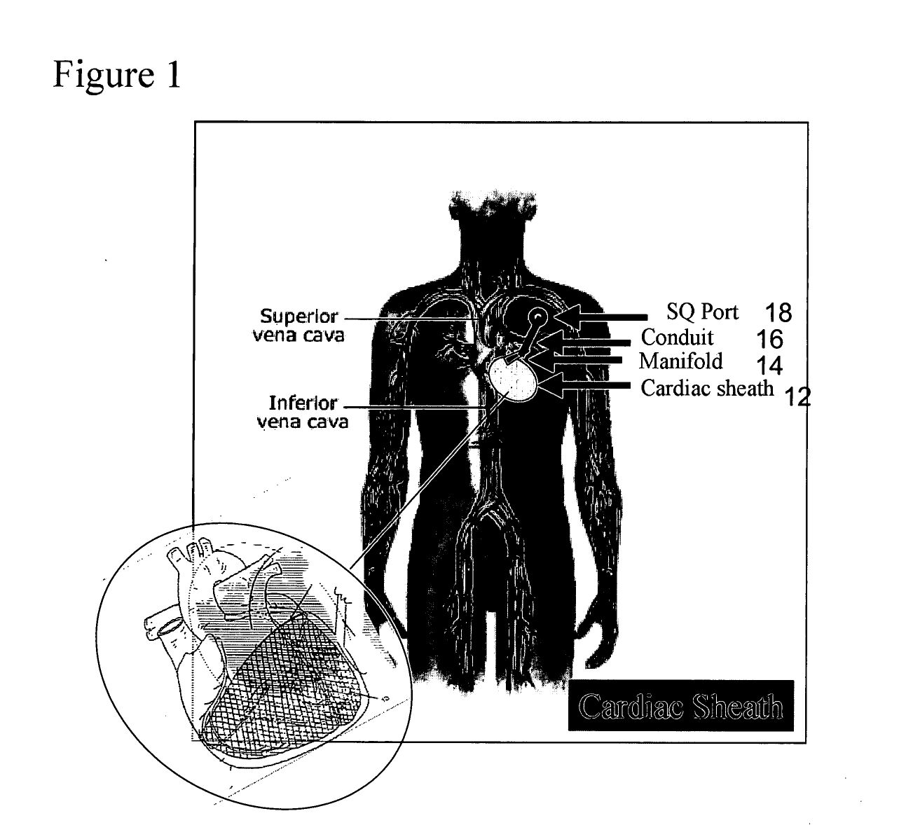

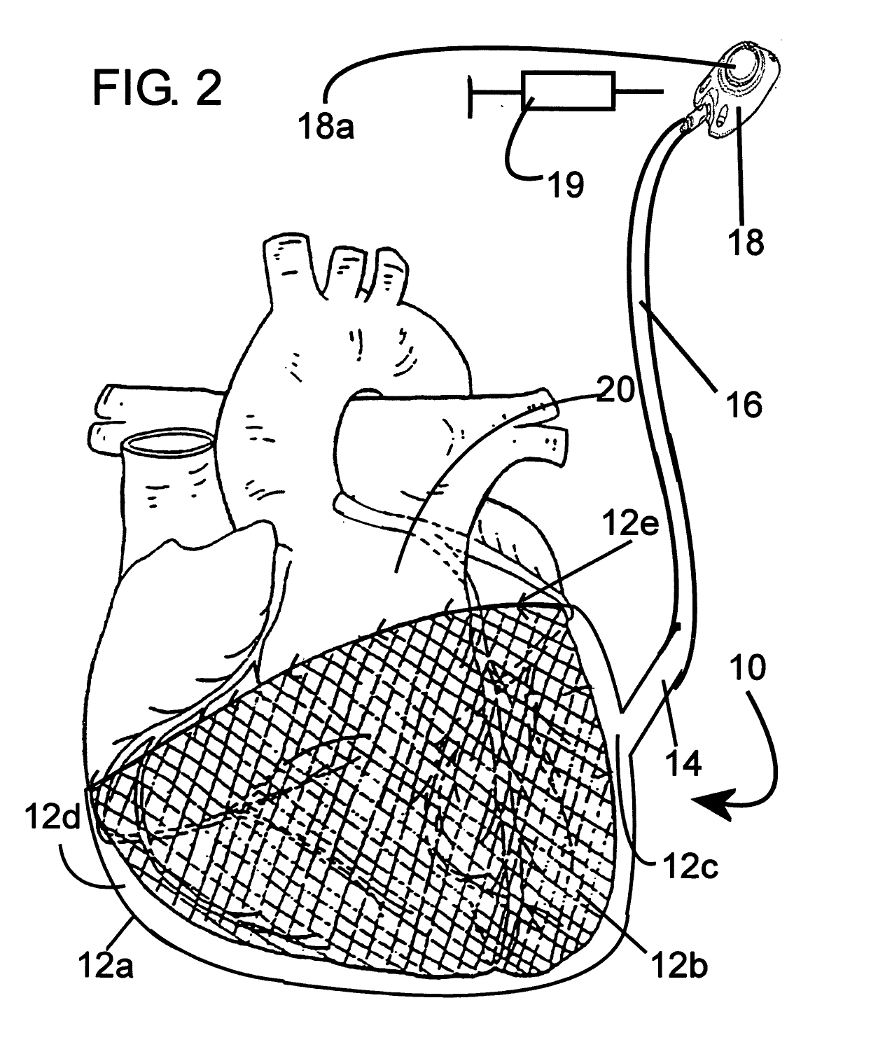

As shown in FIGS. 1 and 2, a medical device 10 can be implanted in humans and provide a mechanism for the delivery of therapeutic agents to the surface of the heart 20 on a long-term basis. The duration of therapy may vary from weeks to years, to life long treatment. Therapeutic agents include but are not limited to cellular therapy, gene therapy, pharmacologic agents, biological agents, drug-coated beads or polymers, innovative medical procedures and the like.

The device 10 generally comprises a sheath 12, manifold 14, conduit 16, and subcutaneous port 18. The sheath 12 can be comprised of an outer membrane 12a, inner membrane 12b, an opening 12c in the outer membrane 12a, a cavity 12d between the inner 12b and outer 12a membranes. The outer membrane 12a of the sheath 12 can be connected to a tube/conduit 16 by means of a manifold 14, which is in turn connected to a subcutaneous port 18 that permits delivery of biological and pharmacologic agents directly to the surface of the heart. This port 18 is easily accessed percutaneosly and may be located in a convenient position on the body such as below the clavicle. After analgesia is applied to the skin overlying the port 18, a needle 19 is inserted percutaneously directly into the port 18 through the port input 18a and the pharmacologic or biologic agent may be delivered over the course of minutes to hours. Once the delivery is completed the needle 19 may be removed and the patient can continue to benefit from the directed therapy until the next delivery is scheduled.

In one embodiment, a portable pump (not shown but known in the art) may be attached to the delivery needle 19 and/or port 18 to provide continuous delivery of the agent. The portable pump may be carried in a fanny pack on the person, and refilled as needed.

The device 10 is designed for the treatment of both ischemic and dilated cardiomyopathy. However, it is not limited to the treatment of ischemic and dilated cardiomyopathy, and can be used to treat a variety of heart disorders and diseases.

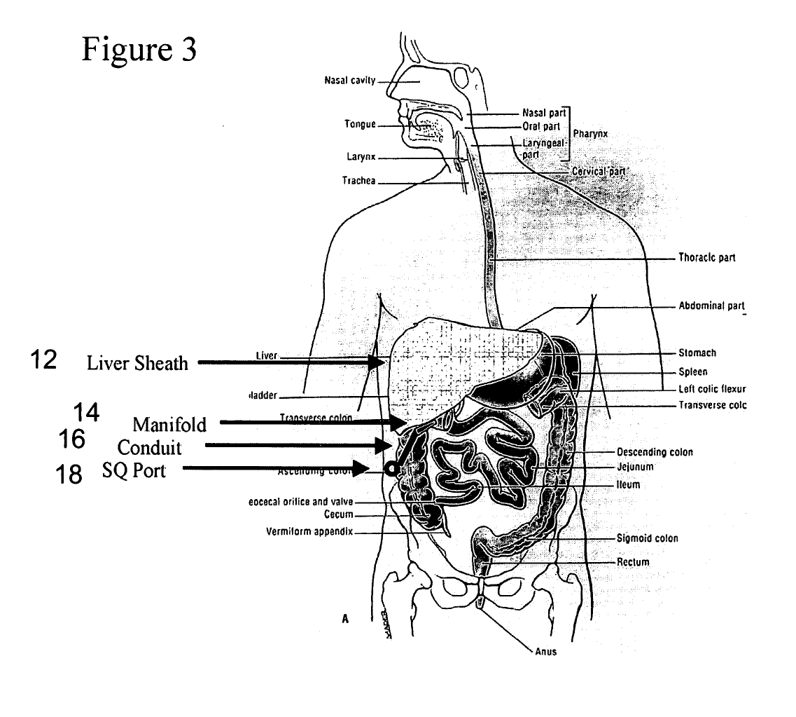

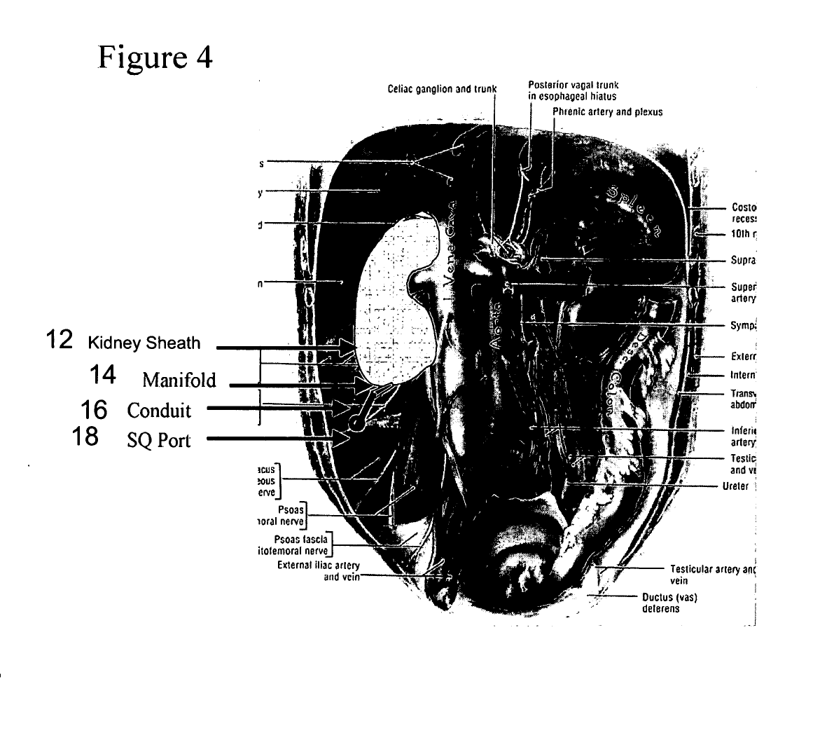

The device 10 may be well-suited for the treatment of the transplanted heart 20 to prevent rejection as well. Furthermore, as shown in FIGS. 3 and 4, the device 10 may also be used for directed therapy to other organs such as the liver, kidney, or bladder. These sheaths 12 would be modified to conform to these particular organs and the implantable port 18 would be located similarly in a nearby subcutaneous position.

The device 10 is made from materials including but not limited to implantable polyester, polyurethane, silicone, other biocompatible polymer, similar compounds, or any combination thereof. In one preferred embodiment, the device 10 is elastic and conforms to the surface of the heart without impeding the epicardial coronary flow, or causing restriction or constriction of the heart. The device 10 of the current invention can be composed of multiple hollow fibers, multiple membrane layers or a combination thereof. In a preferred embodiment, the composition of the device 10 is hollow membrane fibers or membranes that are flexible and biocompatible. The fibers can be arranged in a variety of ways including but not limited to a single layer configuration of microporous fibers, a bi-layer arrangement of fibers, and the like. The current invention, however, is not so limited and can likewise be composed of any desired material necessary to carry out the intended functions.

In one embodiment, only the inner membrane 12b of the sheath 12 is permeable to biological agents, nanoparticles, cells, and pharmacologic agents. The inner membrane supports the ventricles as is taught in the patents and products of Acorn Laboratories, for example, U.S. Pat. No. 6,416,459 herein incorporated by reference as if fully set forth. In another embodiment the outer membrane 12a is comprised of a membrane impermeable to biological agents, cells, and pharmacologic agents. The device 12 can have antimicrobial properties or other desirable properties. Thus, a cavity 12d between the membranes would receive treatment delivered to the sheath 12 and contain this treatment in proximity to the heart 20.

As seen in FIGS. 1-4, the conduit 16 can be composed of materials including but not limited to silicone, rubber, other suitable materials or a combination thereof. The port 18 can be composed of materials including but not limited to biocompatible polyurethane with a central silicone core that connects to the conduit. Further, the port 18 can be accessed by percutaneous needle for intermittent or continuous delivery of biologic or pharmacologic agents. A portable infusion pump can be used to permit ambulation and chronic administration.

The port 18 is connected by a conduit 16 such as a catheter to the sheath that surrounds the heart. This catheter lies in the subcutaneous plane along the antero-lateral chest wall. Preferably, it passes through the intercostal muscles, across the pleural space, and into the pericardial space in the mediastinum. The catheter can be connected to the heart sheath by means of a polypropylene or polyurethane manifold. This portion of the device will deliver the therapeutic agent from the catheter to the sheath effectively.

The device 10 can be inserted in patients with various types of heart disease to administer biologic or pharmacologic agents directly to the heart. In one embodiment, the device 10 may be used as an adjunct to standard therapy such as oral and intravenous medications. In another embodiment it may be used to provide directed therapy of pharmacologic agents that are normally delivered by oral or intravenous routes. The device 10 can be used for patients with ventricular assist device support to improve myocardial recovery. In another embodiment, the current invention can be used for patients after heart transplantation to prevent acute and chronic rejection. In similar fashion, the device may be used with any solid organ to prevent rejection, treat infection, or provide directed therapy.

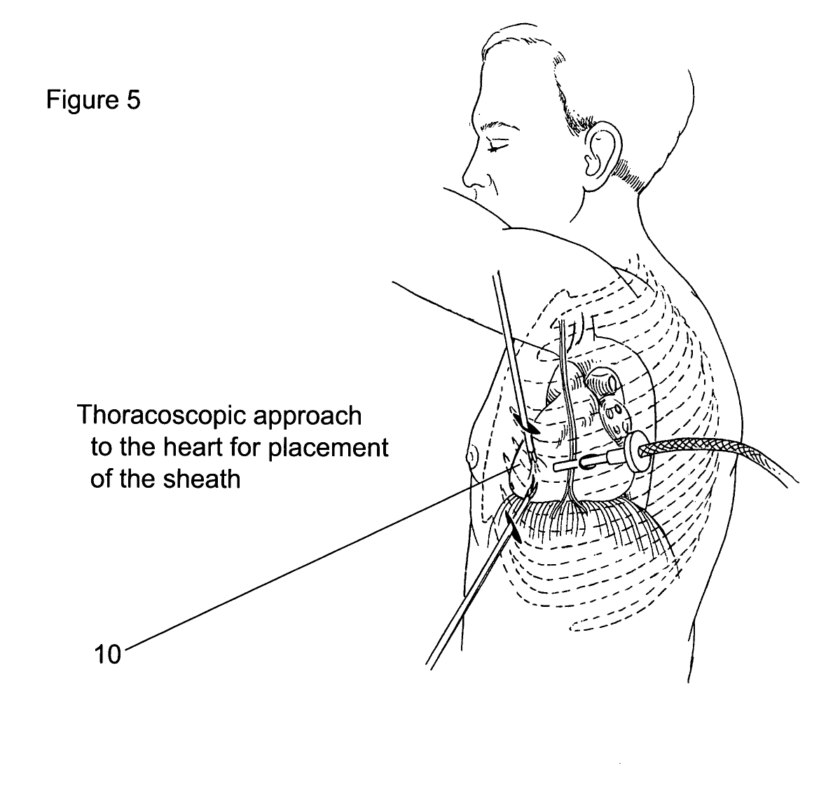

The device 10 can be attached to the heart 20 by open sternotomy or by thoracoscopic access to the mediastinum (See FIG. 5). Insertion can occur at the time of a concomitant procedure such as coronary artery bypass, valve replacement or repair, ventricular assist device insertion, heart transplantation, or alone. In one embodiment, the device can be designed to cover the entire right and left ventricular surface. In one preferred embodiment, the device 10 attaches to the heart 20 by the use of nitinol clips that may be attached directly or by minimally invasive procedures (thoracoscope). In another embodiment, polypropylene sutures 12e or staples may also be used. In still another embodiment, the device 10 may grasp an organ using a drawstring type enclosure that would surround the organ and cinch around various inputs and outputs from the organ.

The device can be manufactured in at least three sizes to fit hearts of varying sizes, but it is not so limited and can be manufactured in a variety of sizes. In one embodiment, it can be shaped to fit the size of the heart encountered by means of cutting the sheath or modeling it to the required size. In another embodiment, the sheath can be modified to accommodate ventricular assist devices or otherwise be modified to accommodate certain portions of the heart. In yet another embodiment, the sheath can be analogously configured to accommodate other body organs or tissues.

As stated previously, the use of biologic agents to regenerate the heart muscle has only recently been made possible with the advances in stem cell therapy and adenoviral gene therapy. Solutions to facilitate delivery to the heart have predominantly been focused on percutaneous trans-venous or trans-arterial approaches. Thus the method, system and device of the current invention addresses the need to effectively deliver therapeutic agents to the surface of the heart or other organs on a long-term basis.

Whereas the present invention has been described in relation to the accompanying drawings, it should be understood that other and further modifications, apart from those shown or suggested herein, may be made within the spirit and scope. It is also intended that all matter contained in the foregoing description or shown in the accompanying drawings shall be interpreted as illustrative rather than limiting.

What is claimed is:

1. A medical device comprising:
a sheath comprising an inner membrane, an outer membrane and an opening in the outer membrane, wherein the sheath surrounds a heart;
a tube operably connected at a first end to the opening in the outer membrane; and
a port operably connected to the tube at the tube's second end, wherein administration of a substance to the port permits the substance to be delivered to the inner membrane of the sheath.

2. The medical device of claim 1, wherein the inner membrane and outer membrane are continuously connected or bonded.

3. The medical device of claim 1, wherein the inner membrane and outer membrane define a space therebetween.

4. The medical device of claim 1, wherein the substance is a therapeutic agent.

5. The medical device of claim 1, wherein the outer membrane is impermeable or substantially impermeable to the substance.

6. The medical device of claim 5, wherein the inner membrane is permeable to the substance.

7. The medical device of claim 1, wherein the port is located under skin, whereby delivery of the substance to the port is effectuated by injection through the skin.

8. A method for delivering a therapeutic agent to a heart comprising the steps of:
surrounding a heart with a sheath having an inner portion, an outer portion and an opening in the outer portion;
connecting a tube at a first end to the outer portion of the sheath;
connecting a port having a port opening to the tube at a second end, wherein the port opening is underneath skin; and
administering the therapeutic agent by injection through the skin into the port opening.

9. A medical device comprising:
a sheath comprising an inner membrane, an outer membrane and an opening in the outer membrane, the inner membrane and outer membrane defining a space, wherein the sheath surrounds a heart;
a tube operably connected at a first end to the opening in the outer membrane; and
a port operably connected to the tube at the tube's second end, wherein administration of a therapy to the port effectuates deliver of the therapy to the space.

10. The medical device of claim 9, wherein the therapy comprises cellular therapy.

11. The medical device of claim 9, wherein the therapy comprises gene therapy.

12. The medical device of claim 9, wherein the therapy comprises pharmacologic agents.

13. The medical device of claim 9, wherein the therapy comprises biological agents.

14. The medical device of claim 9, wherein the therapy comprises drug-coated polymers.

15. The medical device of claim 14, wherein the polymers comprise beads.

16. The medical device of claim 9, wherein the outer membrane is impermeable or substantially impermeable to the therapy.

17. The medical device of claim 9, wherein the inner membrane is permeable to the therapy.

18. The medical device of claim 9, wherein the port is positioned adjacent to a person's clavicle.

19. A method of delivering a therapy to a heart comprising the steps of:
surrounding the heart with a sheath comprising an inner membrane, an outer membrane and an opening in the outer membrane, the inner membrane and outer membrane defining a space;
connecting a tube at a first end to the opening in the outer membrane; connecting a port at to the tube at the tube's second end; and
administering a therapy to the port for delivery of the therapy to the space.

20. A method of delivering a therapy to an organ comprising the steps of:
surrounding the organ with a sheath comprising an inner membrane, an outer membrane and an opening in the outer membrane, the inner membrane and outer membrane defining a cavity;
connecting a tube at a first end to the opening in the outer membrane; connecting a port to the tube at the tube's second end; and
administering a therapy to the port for delivery of the therapy to the cavity.

21. The method of claim 20, further comprising inserting a needle percutaneously into the port to inject the therapy into the port.

22. The method of claim 21, further comprising connecting a pump operably to the needle, wherein the pump is capable of effectuating continual delivery of the therapy.

23. The method of claim 22, wherein the organ is a transplanted organ.

24. The method of claim 23, wherein the therapy comprises immunosuppression agents.

25. A method of delivering a therapy to human tissue comprising the steps of:
surrounding the human tissue with a sheath comprising an inner membrane, an outer membrane and an opening in the outer membrane, the inner membrane and outer membrane defining a cavity;
connecting a tube at a first end to the opening in the outer membrane; connecting a port to the tube at the tube's second end; and
administering a therapy to the port for delivery of the therapy to the cavity.

26. A medical device comprising:
a sheath comprising a membrane, the membrane having an opening and defining a cavity, wherein the sheath surrounds a heart;
a tube operably connected at a first end to the opening; and
a port operably connected to the tube at the tube's second end, wherein administration of a substance to the port permits the substance to be delivered to the cavity of the sheath.
